(19) United States
(12) Patent Application Publication
(10) Pub. No.: US 2004/0115242 A1
(43) Pub. Date: Jun. 17, 2004

(54) Antimicrobial contact lenses and methods for their production

(76) Inventors: Ann-Marie W. Meyers, Jacksonville, FL (US); Frank L. Neely, Jacksonville, FL (US); John B. Enns, Jacksonville, FL (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/703,770

(22) Filed: Nov. 7, 2003

Related U.S. Application Data
(63) Continuation-in-part of application No. 10/028,400, filed on Dec. 20, 2001.

(51) Int. Cl. A61K 33/38; A61K 9/00

(52) U.S. Cl. 424/429; 424/618

(57) ABSTRACT

This invention relates to a method for forming antimicrobial lenses comprising silver and a ligand monomer comprising;

(a) curing a reactive monomer mixture comprising at least one lens forming component and at least one ligand monomer under conditions sufficient to provide a reactivity ratio of the ligand monomer to at least one major lens forming component of at least about 0.45; and

(b) treating said lens with a silver solution to form an antimicrobial lens comprising silver in an amount which is at least about 80% of target silver concentration.

### RELATED INVENTIONS

This patent application claims priority from U.S. Ser. No. 10/028,400, filed on Dec. 20, 2001.

### FIELD OF THE INVENTION

This invention relates to contact lenses having antimicrobial properties as well as methods of their production, use, and storage.

### BACKGROUND OF THE INVENTION

Contact lenses have been used commercially to improve vision since the 1950s. The first contact lenses were made of hard materials. Although these lenses are currently used, they are not suitable for all patients due to their poor initial comfort and their relatively low permeability to oxygen. Later developments in the field gave rise to soft contact lenses, based upon hydrogels, which are extremely popular today. Many users find soft lenses are more comfortable, and increased comfort levels allow soft contact lens users to wear their lenses for far longer hours than users of hard contact lenses.

Despite this advantage, the extended use of the lenses can encourage the buildup of bacteria or other microbes, particularly, Pseudomonas aeruginosa, on the surfaces of soft contact lenses. The build-up of bacteria or other microbes is not unique to soft contact lens wearers and may occur during the use of hard contact lenses as well.

Therefore, there is a need to produce contact lenses that inhibit the growth of bacteria or other microbes and/or the adhesion of bacteria or other microbes on the surface of contact lenses. Further there is a need to produce contact lenses which do not promote the adhesion and/or growth of bacteria or other microbes on the surface of the contact lenses. Also there is a need to produce contact lenses that inhibit adverse responses in the eye related to the growth of bacteria or other microbes.

Others have recognized the need to produce soft contact lenses that inhibit the growth of bacteria. In U.S. Pat. No. 5,213,801, the production of an antibacterial contact lens is disclosed, where an antibacterial metal ceramic material within a soft contact lens is incorporated into a contact lens. This procedure contains a number of steps and may not be suitable for producing all types of lenses in a production environment. The steps include making a silver ceramic material that is fine enough to be used in a contact lens and then forming the lens with the powdered ceramic. However, lenses containing these types of materials often lack the clarity required by contact lens users.

Although these methods and lenses are known, other contact lenses that inhibit the growth and/or adhesion of bacteria or other microbes and are of sufficient optical clarity, as well as methods of making those lenses are still needed. It is this need, which this invention seeks to meet.

### DESCRIPTION OF THE FIGURES

FIG. 1 is a graph of the normalized concentration of 2-hydroyxethyl methacrylate and cystamine as a function of reaction time at 1 mW/cm² and 0.45 wt % photoinitiator concentration.

FIG. 2 is a graph of the normalized concentration of 2-hydroyxethyl methacrylate and cystamine as a function of reaction time at 6 mW/cm² and 0.9 wt % photoinitiator concentration.

FIG. 3 is a graph of the normalized concentration of 2-hydroyxethyl methacrylate and cystamine as a function of reaction time at 6 mW/cm² and 1.35 wt % photoinitiator concentration.

FIG. 4 is a graph of the percent silver incorporated into a lens as a function of the reactivity ratio of cystamine to 2-hydroyxethyl methacrylate at different photoinitiator concentrations and radiation intensities.

### DETAILED DESCRIPTION OF THE INVENTION

This invention includes a process for making an antimicrobial lens having consistent quantities of silver bound thereto. Specifically, the process of the present invention comprises curing a monomer mixture comprising lens forming components and at least one ligand monomer under conditions sufficient to provide a relative reactivity ratio of the ligand monomer to a major lens forming component of at least about 0.45 and contacting said lens with a silver containing solution to form an antimicrobial lens comprising silver ions in an amount greater than about 80% of a target silver concentration.

The lenses of the present invention comprise, consist essentially of, or consist of, silver and a polymer formed from a reaction mixture comprising at least lens forming component and at least one ligand monomer. As used herein a ligand monomer is a monomer which is capable of reversibly binding cations, particularly antimicrobial cations and most particularly silver. Specific ligand monomers include those of Formulae I, II, III and IV

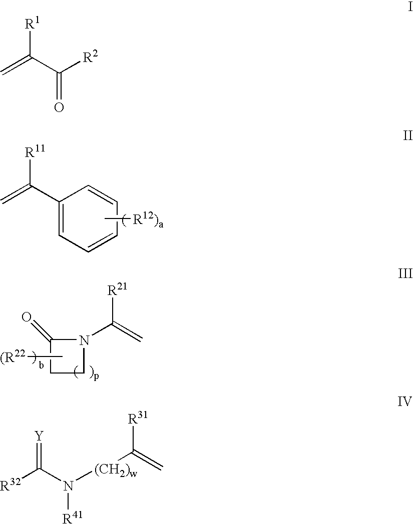

wherein

R¹ is hydrogen or C₁₋₆-alkyl;

R² is —OR³, —NH—R³—S—(CH₂)_{d}—R³, or —(CH₂)_{d}—R³, wherein

d is 0-8;

R³is substituted C₁₋₆-alkyl

where the alkyl substituents are selected from one or more members of the group consisting of carboxylic acid, sulfonic acid, phosphonic acid, amine, amidine, acetamide, nitrile, thiol, C₁₋₆alkyldisulfide,

C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea, and substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;

—(CR⁴R⁵)_{q}—(CHR⁶)ₘ—SO₃H

wherein R⁴, R⁵, and R⁶ are independently selected from the group consisting of hydrogen, halogen, hydroxyl, and C₁₋₆alkyl,

q is 1-6, and m is 0-6;

—(CH₂)ₙ—S—S—(CH₂)ₓNH—C(O)CR⁷CH₂,

wherein R⁷ is hydrogen or C₁₋₆alkyl,

n is 1-6, and x is 1-6;

—(CR⁸R⁹)ₜ—(CHR¹⁰)ᵤ—P(O)(OH)₂

wherein R⁸, R⁹, and R¹⁰ are independently selected from the group consisting of hydrogen, halogen, hydroxyl, and C, ₆alkyl,

t is 1-6, and

u is 0-6;

phenyl; benzyl; pyridinyl; pyrimidinyl; pyrazinyl;

benzimidazolyl; benzothiazolyl; benzotriazolyl;

naphthaloyl; quinolinyl; indolyl; thiadiazolyl; triazolyl;

4-methylpiperidin-1-yl; 4-methylpiperazin-1-yl;

substituted phenyl; substituted benzyl; substituted pyridinyl; substituted pyrimidinyl; substituted pyrazinyl;

substituted benzimidazolyl; substituted benzothiazolyl;

substituted benzotriazolyl; substituted naphthaloyl;

substituted quinolinyl; substituted indolyl; substituted thiadiazolyl; substituted triazolyl; substituted 4-methylpiperidin-1-yl;

or substituted 4-methylpiperazin-1-yl,

wherein the substituents are selected from one or more members of the group consisting of C₁₋₆-alkyl, haloC₁₋₆alkyl, halogen, sulfonic acid, phosphonic acid, hydroxyl, carboxylic acid, amine, amidine,

N-(2-aminopyrimidine)sulfonyl,

N-(aminopyridine)sulfonyl, N-(aminopyrazine)sulfonyl,

N-(2-aminopyrimidine)carbonyl,

N-(aminopyridine)carbonyl, N-(aminopyrazine)carbonyl,

N-(2-aminopyrimidine)phosphonyl,

N-(2-aminopyridine)phosphonyl,

N-(aminopyrazine)phosphonyl,

N-(aminobenzimidazolyl)sulfonyl,

N-(aminobenzothiazolyl)sulfonyl,

N-(aminobenzotriazolyl)sulfonyl,

N-(aminoindolyl)sulfonyl, N-(aminothiazolyl)sulfonyl,

N-(aminotriazolyl)sulfonyl,

N-(amino-4-methylpiperidinyl)sulfonyl,

N-(amino-4-methylpiperazinyl)sulfonyl,

N-(aminobenzimidazolyl)carbonyl,

N-(aminobenzothiazolyl)carbonyl,

N-(aminobenzotriazolyl)carbonyl,

N-(aminoindolyl )carbonyl, N-(aminothiazolyl)carbonyl,

N-(aminotriazolyl)carbonyl,

N-(amino-4-methylpiperidinyl)carbonyl,

N-(amino-4-methylpiperazinyl)carbonyl,

N-(2-aminobenzimidazolyl)phosphonyl,

N-(2-aminobenzothiazolyl)phosphonyl,

N-(2-aminobenzotriazolyl)phosphonyl,

N-(2-aminoindolyl)phosphonyl,

N-(2-aminothiazolyl)phosphonyl,

N-(2-aminotriazolyl)phosphonyl,

N-(amino-4-methylpiperidinyl) phosphonyl,

N-(amino-4-methylpiperazinyl) phosphonyl, acetamide,

nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea

wherein the C₁₋₆alkyldisulfide, phenyldisulfide, C₁₋₆alkylurea, C₁₋₆-alkylthiourea, phenylurea, and phenylthiourea substituents are selected from the group consisting of C₁-alkyl, haloC₁₋₆alkyl, halogen, hydroxyl, carboxylic acid, sulfonic acid, phosphonic acid, amine, amidine, acetamide, and nitrile;

a is 1-5;

R¹¹ is hydrogen or C₁₋₆alkyl;

R¹² is hydroxyl, sulfonic acid, phosphonic acid, carboxylic acid, acetamide, thioC₁₋₆alkylcarbonyl, C₁₋₆-alkyldisulfide, C₁₋₆alkylsulfide, phenyl disulfide, urea, C₁₋₆-alkylurea, phenylurea, thiourea, C₁₋₆alkylthiourea, phenylthiourea, —OR¹³, —NH—R¹³, —S—(CH₂)_{d}—R¹³, —(CH₂)_{d}—R¹³, —C(O)NH—(CH₂)_{d}—R¹³, —C(O)—(CH₂)_{d}—R¹³, substituted C₁₋₆alkyldisulfide, substituted phenyldisulfide, substituted C₁₋₆alkylurea, substituted phenylurea, substituted phenylthiourea or substituted C₁₋₆alkylthiourea wherein the substituents are selected from the group consisting of C₁₋₆-alkyl, haloC₁₋₆alkyl, halogen, hydroxyl, carboxylic acid, sulfonic acid, phosphonic acid, amine, amidine, acetamide, and nitrile;

where

d is 0-8;

R¹³ is thioC₁₋₆alkylcarbonyl;

substituted C₁₋₆-alkyl

where the alkyl substituents are selected from one or more members of the group consisting of hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea and substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;

—(CR¹⁴R¹⁵)_{q}—(CHR¹⁶)ₘ—SO₃H

where R¹⁴, R¹⁵, and R¹⁶ are independently selected from the group consisting of hydrogen, halogen, hydroxyl, and C₁₋₆alkyl,

q is 1-6, and m is 0-6;

—(CH₂)ₙ—S—S—(CH₂)ₓNH—C(O)CR¹⁷CH₂,

where R¹⁷ is hydrogen or C₁₋₆alkyl,

n is 1-6, and x is 1-6;

—(CR¹⁸R¹⁹)ₜ—(CHR²)ᵤ—P(O)(OH)₂

where R^{18,} R¹⁹, and R²⁰ are independently selected from the group consisting of hydrogen, halogen, hydroxyl, and C₁₋₆alkyl,

t is 1-6, and u is 0-6;

phenyl; benzyl; pyridinyl; pyrimidinyl; pyrazinyl;

benzimidazolyl; benzothiazolyl; benzotriazolyl;

naphthaloyl; quinolinyl; indolyl; thiadiazolyl; triazolyl;

4-methylpiperidin-1-yl; 4-methylpiperazin-1-yl;

substituted phenyl; substituted benzyl; substituted pyridinyl; substituted pyrimidinyl; substituted pyrazinyl;

substituted benzimidazolyl; substituted benzothiazolyl;

substituted benzotriazolyl; substituted naphthaloyl;

substituted quinolinyl; substituted indolyl; substituted thiadiazolyl; substituted triazolyl; substituted 4-methylpiperidin-1-yl; or substituted 4-methylpiperazin-1-yl

wherein the substituents are selected from one or more members of the group consisting of C₁₋₆alkyl, haloC₁₆alkyl, halogen, sulfonic acid, phosphonic acid, hydroxyl, carboxylic acid, amine, amidine,

N-(2-aminopyrimidine)sulfonyl,

N-(aminopyridine)sulfonyl, N-(aminopyrazine)sulfonyl,

N-(2-aminopyrimidine)carbonyl,

N-(aminopyridine)carbonyl, N-(aminopyrazine)carbonyl,

N-(2-aminopyrimidine)phosphonyl,

N-(2-aminopyridine)phosphonyl,

N-(aminopyrazine)phosphonyl,

N-(aminobenzimidazolyl)sulfonyl,

N-(aminobenzothiazolyl)sulfonyl,

N-(aminobenzotriazolyl)sulfonyl,

N-(aminoindolyl)sulfonyl, N-(aminothiazolyl)sulfonyl,

N-(aminotriazolyl)sulfonyl,

N-(amino-4-methylpiperidinyl)sulfonyl,

N-(amino-4-methylpiperazinyl)sulfonyl,

N-(aminobenzimidazolyl)carbonyl,

N-(aminobenzothiazolyl)carbonyl,

N-(aminobenzotriazolyl)carbonyl,

N-(aminoindolyl)carbonyl, N-(aminothiazolyl)carbonyl,

N-(aminotriazolyl)carbonyl,

N-(amino-4-methylpiperidinyl)carbonyl,

N-(amino-4-methylpiperazinyl)carbonyl,

N-(2-aminobenzimidazolyl)phosphonyl,

N-(2-aminobenzothiazolyl)phosphonyl,

N-(2-aminobenzotriazolyl)phosphonyl,

N-(2-aminoindolyl)phosphonyl,

N-(2-aminothiazolyl)phosphonyl,

N-(2-aminotriazolyl)phosphonyl,

N-(amino-4-methylpiperidinyl) phosphonyl,

N-(amino-4-methylpiperazinyl) phosphonyl, acetamide,

nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;

b is 1-5; p is 1-5;

R²¹ is hydrogen;

R²² is hydroxyl, sulfonic acid, phosphonic acid, carboxylic acid, thioC₁₋₆-alkylcarbonyl, thioC₁₋₆alkylaminocarbonyl, C₁₋₆alkyldisulfide, phenyldisulfide, —C(O)NH(CH₂)₁₋₆—SO₃H, —C(O)NH(CH₂)₁₋₆—P(O)(OH)₂, —OR²³, —NH—R²³, —C(O)NH—(CH₂)_{d}—R^{23′}—S—(CH₂)_{d}—R²³, —(CH₂)_{d}—R²³, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted, C₁-alkylthiourea substituted phenylurea or substituted phenylthiourea wherein the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile,

where

d is 0-8;

R²³ is thioC₁₋₆alkylcarbonyl,

C₁₋₆alkyl,

substituted C₁₋₆-alkyl

where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆-alkylurea, phenylurea, thiourea, C₁₋₆-alkylthiourea, phenylthiourea, substituted C₁₋₆alkyldisulfide, substituted phenyldisulfide, substituted C₁₋₆alkylurea, substituted phenylurea, substituted C₁₋₆-alkylthiourea, and substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;

—(CR²⁴R²⁵)_{q}—(CHR²⁶)ₘ—SO₃H

where R²⁴, R²⁵, and R²⁶ are independently selected from the group consisting of hydrogen, halogen, hydroxyl, and C₁₋₆alkyl,

q is 1-6, and m is 0-6

—(CH₂)ₙ—S—S—(CH₂)ₓNH—C(O)CR²⁷CH₂,

where R²⁷ is hydrogen or C₁₋₆alkyl,

n is 1-6, and x is 1-6;

—(CR²⁸R²⁹)ₜ—(CHR³⁰)ᵤ—P(O)(OH)₂

where R²⁸, R²⁹, and R³⁰ are independently selected from the group consisting of hydrogen, halogen, hydroxyl, and C₁₋₆alkyl,

t is 1-6, and u is 0-6;

phenyl; benzyl; pyridinyl; pyrimidinyl; pyrazinyl;

benzimidazolyl; benzothiazolyl; benzotriazolyl; naphthaloyl;

quinolinyl; indolyl; thiadiazolyl; triazolyl;

4-methylpiperidin-1-yl; 4-methylpiperazin-1-yl; substituted

phenyl; substituted benzyl; substituted pyridinyl;

substituted pyrimidinyl; substituted pyrazinyl; substituted benzimidazolyl; substituted benzothiazolyl; substituted benzotriazolyl; substituted naphthaloyl; substituted quinolinyl; substituted indolyl; substituted thiadiazolyl;

substituted triazolyl; substituted 4-methylpiperidin-1-yl; or

substituted 4-methylpiperazin-1-yl,

wherein the substituents are selected from one or more members of the group consisting of C₁₋₆alkyl,

haloC₁₋₆alkyl, halogen, sulfonic acid, phosphonic acid,

hydroxyl, carboxylic acid, amine, amidine,

N-(2-aminopyrimidine)sulfonyl,

N-(aminopyridine)sulfonyl, N-(aminopyrazine)sulfonyl,

N-(2-aminopyrimidine)carbonyl,

N-(aminopyridine)carbonyl, N-(aminopyrazine)carbonyl,

N-(2-aminopyrimidine)phosphonyl,

N-(2-aminopyridine)phosphonyl,

N-(aminopyrazine)phosphonyl,

N-(aminobenzimidazolyl)sulfonyl,

N-(aminobenzothiazolyl)sulfonyl,

N-(aminobenzotriazolyl)sulfonyl,

N-(aminoindolyl)sulfonyl, N-(aminothiazolyl)sulfonyl,

N-(aminotriazolyl)sulfonyl,

N-(amino-4-methylpiperidinyl)sulfonyl,

N-(amino-4-methylpiperazinyl)sulfonyl,

N-(aminobenzimidazolyl)carbonyl,

N-(aminobenzothiazolyl)carbonyl,

N-(aminobenzotriazolyl)carbonyl,

N-(aminoindolyl)carbonyl, N-(aminothiazolyl)carbonyl,

N-(aminotriazolyl)carbonyl,

N-(amino-4-methylpiperidinyl)carbonyl,

N-(amino-4-methylpiperazinyl)carbonyl,

N-(2-aminobenzimidazolyl)phosphonyl,

N-(2-aminobenzothiazolyl)phosphonyl,

N-(2-aminobenzotriazolyl)phosphonyl,

N-(2-aminoindolyl)phosphonyl,

N-(2-aminothiazolyl)phosphonyl,

N-(2-aminotriazolyl)phosphonyl,

N-(amino-4-methylpiperidinyl) phosphonyl,

N-(amino-4-methylpiperazinyl) phosphonyl, acetamide,

nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;

w is 0-1; Y is oxygen or sulfur;

R³¹ is hydrogen or C₁₋₆alkyl;

R³² is hydroxyl, sulfonic acid, phosphonic acid, carboxylic acid, thioC₁₋₆alkylcarbonyl, thioC₁₋₆alkylaminocarbonyl, —C(O)NH—(CH₂)_{d}—R³³, —O—R³³, —NH—R³³, —S—(CH₂)_{d}—R³³, —(CH₂)_{d}—R³³, C₁₋₆alkyldisulfide, phenyldisulfide, urea, C₁₋₆alkylurea, phenylurea, thiourea, C₁₋₆alkylthiourea, phenylthiourea, C₁₆alkylamine, phenylamine, substituted C₁₋₆alkyldisulfide, substituted phenyldisulfide, substituted phenylurea, substituted C₁₋₆alkylamine, substituted phenylamine, substituted phenylthiourea, substituted C₁₋₆alkylurea or substituted C₁₋₆alkylthiourea wherein the substitutents are selected from the group consisting of C₁₆alkyl, haloC₁₋₆alkyl, halogen, hydroxyl, carboxylic acid, sulfonic acid, phosphonic acid, amine, amidine, acetamide, and nitrile where

d is 0-8;

R³³ is thioC₁₋₆alkylcarbonyl,

C₁₋₆alkyl,

substituted C₁₋₆alkyl

where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea or substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;

—(CR³⁴R³⁵)_{q}—(CHR³⁶)ₘ—SO₃H

where R³⁴, R³⁵, and R³⁶ are independently selected from the group consisting of hydrogen, halogen, hydroxyl, and C₁₋₆alkyl,

q is 1-6, and m is 0-6;

—(CH₂)ₙ—S—S—(CH₂)ₓNH—C(O)CR³⁷CH₂,

where R³⁷ is hydrogen or C₁₋₆alkyl,

n is 1-6, and x is 1-6;

—(CR³⁸R³⁹)ₜ—(CHR⁴⁰)ᵤ—P(O)(OH)₂

where R³⁸, R³⁹, and R⁴⁰ are independently selected from the group consisting of hydrogen, halogen, hydroxyl, and C₁₆alkyl,

t is 1-6, and u is 0-6;

phenyl; benzyl; pyridinyl; pyrimidinyl; pyrazinyl;

benzimidazolyl; benzothiazolyl; benzotriazolyl;

naphthaloyl; quinolinyl; indolyl; thiadiazolyl; triazolyl;

4-methylpiperidin-1-yl; 4-methylpiperazin-1-yl; substituted phenyl; substituted benzyl; substituted pyridinyl;

substituted pyrimidinyl; substituted pyrazinyl; substituted benzimidazolyl; substituted benzothiazolyl; substituted benzotriazolyl; substituted naphthaloyl; substituted quinolinyl; substituted indolyl; substituted thiadiazolyl;

substituted triazolyl; substituted 4-methylpiperidin-1-yl; or

substituted 4-methylpiperazin-1-yl,

wherein the substituents are selected from one or more members of the group consisting of C₁₋₆alkyl, haloC₁₋₆alkyl, halogen, sulfonic acid, phosphonic acid, hydroxyl, carboxylic acid, amine, amidine,

N-(2-aminopyrimidine)sulfonyl,

N-(aminopyridine)sulfonyl, N-(aminopyrazine)sulfonyl,

N-(2-aminopyrimidine)carbonyl,

N-(aminopyridine)carbonyl, N-(aminopyrazine)carbonyl,

N-(2-aminopyrimidine)phosphonyl,

N-(2-aminopyridine)phosphonyl,

N-(aminopyrazine)phosphonyl,

N-(aminobenzimidazolyl)sulfonyl,

N-(aminobenzothiazolyl)sulfonyl,

N-(aminobenzotriazolyl)sulfonyl,

N-(aminoindolyl)sulfonyl, N-(aminothiazolyl)sulfonyl,

N-(aminotriazolyl)sulfonyl,

N-(amino-4-methylpiperidinyl)sulfonyl,

N-(amino-4-methylpiperazinyl)sulfonyl,

N-(aminobenzimidazolyl)carbonyl,

N-(aminobenzothiazolyl)carbonyl,

N-(aminobenzotriazolyl)carbonyl,

N-(aminoindolyl)carbonyl, N-(aminothiazolyl)carbonyl,

N-(aminotriazolyl)carbonyl,

N-(amino-4-methylpiperidinyl)carbonyl,

N-(amino-4-methylpiperazinyl)carbonyl,

N-(2-aminobenzimidazolyl)phosphonyl,

N-(2-aminobenzothiazolyl)phosphonyl,

N-(2-aminobenzotriazolyl)phosphonyl,

N-(2-aminoindolyl)phosphonyl,

N-(2-aminothiazolyl)phosphonyl,

N-(2-aminotriazolyl)phosphonyl,

N-(amino-4-methylpiperidinyl) phosphonyl,

N-(amino-4-methylpiperazinyl) phosphonyl, acetamide,

nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and,substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;

R⁴¹ is hydrogen, C₁₋₆alkyl, phenyl, C₁₋₆alkylcarbonyl, phenylcarbonyl, substituted C₁₋₆alkyl, substituted phenyl, substituted C₁₋₆alkylcarbonyl or substituted phenylcarbonyl,

wherein

the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile.

Preferred ligand monomers include monomers of Formula I where

R¹ is hydrogen or C₁₋₃alkyl;

R² is NH—R³; d is 0

R³ is substituted phenyl, —(CR⁴R⁵)_{q}—(CHR⁶)ₘ—SO₃H,

—(CR⁸R⁹)ₜ—(CHR¹⁰)ᵤ—(O)(OH)₂, or —(CH₂)ₙ—S—S—(CH₂)ₓNH—C(O)CR⁷CH₂;

R⁴⁻⁶ are independently hydrogen or C₁₋₃alkyl;

q is 1-3; m is 1-3;

R⁷⁻¹⁰ are independently hydrogen or C₁₋₃alkyl;

t is 1-3; u is 1-3; n is 2-4; and x is 2-4.

More preferred ligand monomers include monomers of Formula I where

R¹ is hydrogen or methyl; R² is NH—R³;

R³ is —(CR⁴R⁵)_{q}—(CHR⁶)ₘ—SO₃H, —(CR⁸R⁹)ₜ—(CHR¹⁰)ᵤ—P(O)(OH)₂ or

—(CH₂)ₙ—S—S—(CH₂)ₓNH—C(O)CHR⁷CH₂;

R⁴⁻⁶ and R⁸⁻¹⁰ are independently hydrogen or methyl;

q is 1-2; m is 1-2; R⁷ is hydrogen;

t is 1; u is 1-2; n is 2-3; and x is 2-3.

The most preferred ligand monomers of Formula I include

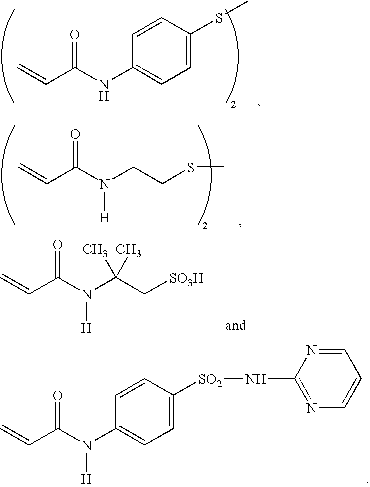

The preferred monomers of Formula II include monomers where a is 1-2; R¹¹ is hydrogen or C₁₋₃alkyl;

R¹² is sulfonic acid, carboxylic acid, phosphonic acid, C₁₋₆alkyldisulfide, C₁₋₆alkylsulfide, phenyldisulfide, substiuted phenyldisulfide or NH—R¹³;

R¹³ is thioC₁₋₆alkylcarbonyl.

The most preferred monomers of Formula II include the following monomers

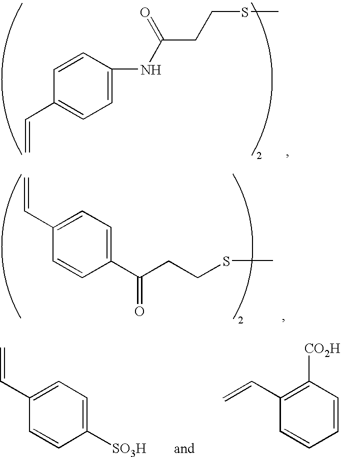

The preferred monomers of Formula III include monomers where p is 1-3; b is 1-2; R is hydrogen;

R²² is sulfonic acid, phosphonic acid, carboxylic acid, thioC₁₋₆alkylcarbonyl, thioC₁₋₆alkylaminocarbonyl, C₁₋₆alkyldisulfide, C₁₋₆alkylsulfide, phenyldisulfide, substiuted phenyldisulfide, H₃OS—(CH₂)₁₋₆NHC(O) or (HO)₂(O)P—(CH₂)₁₋₆NHC(O)—.

The most preferred monomers of Formula III include the following monomers

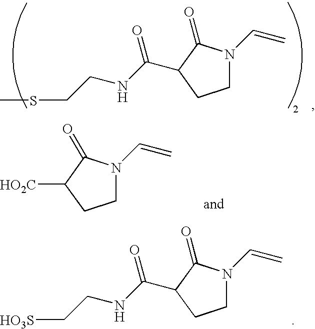

The preferred monomers of Formula IV include monomers where w is 0-1; R³¹ is hydrogen; R³² is amine, C₁₋₃alkylamine, phenylamine, substituted phenylamine, thioC₁₋₃alkylcarbonyl; R⁴¹ is hydrogen.

The most preferred monomers of Formula IV include the following monomers

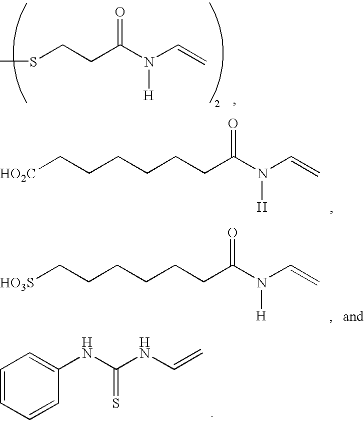

As used herein, the term “lens” refers to opthalmic devices that reside in, or on the eye. These devices can provide optical correction, drug delivery or may be cosmetic. The term lens includes but is not limited to soft contact lenses, hard contact lenses, intraocular lenses, overlay lenses, ocular inserts, and optical inserts. Soft contact lenses are made from silicone elastomers or hydrogels, which include but are not limited to silicone hydrogels and fluorohydrogels. These hydrogels contain hydrophobic and/or hydrophilic monomers that are covalently bound to one another in the cured lens. As used herein the term “polymers” means copolymers, homopolymers, or mixtures thereof.

In the present invention the lens forming components and the ligand monomer are combined and cured under conditions sufficient to provide a relative reactivity ratio of the ligand monomer to at least one major lens forming component of at least about 0.45. Suitable lens forming components are known in the art and include acrylic- or vinyl-containing monomers, hydrophobic monomers and macromers internal wetting agents and compatibilizing monomers and macromers, initiators, UV absorbing compounds, visibility tints, crosslinkers and the like. Acrylic-containing monomers contain the acrylic group: (CH₂═CRCOX) wherein R is H or CH₃, and X is O or N, polymerize readily and include, but are not limited to N,N-dimethyl acrylamide (DMA), 2-hydroxyethyl methacrylate (HEMA), glycerol methacrylate, 2-hydroxyethyl methacrylamide, polyethyleneglycol monomethacrylate, methacrylic acid and acrylic acid.

Vinyl-containing monomers contain the vinyl grouping (—CH═CH₂), and include but are not limited to monomers such as N-vinyl lactams (e.g. NVP), N-vinyl-N-methyl acetamide, N-vinyl-N-ethyl acetamide, N-vinyl-N-ethyl formamide, N-vinyl formamide, with NVP being preferred.

As used herein the term “silicone containing compatibilizing component” means reaction components which contain at least one silicone group and at least one hydroxyl group. Such components have been disclosed in U.S. Ser. Nos. 10/236,538 and 10/236,762.

Silicone-containing components contain at least one [—Si—O—Si] group, and at least one polymerizable functional group in a monomer, macromer or prepolymer. Preferably, the Si and attached O are present in the silicone-containing component in an amount greater than 20 weight percent, and more preferably greater than 30 weight percent of the total molecular weight of the silicone-containing component. Examples of silicone-containing components which are useful in this invention may be found in U.S. Pat. Nos. 3,808,178; 4,120,570; 4,136,250; 4,153,641; 4,740,533; 5,034,461 and 5,070,215, and EP080539.

Suitable soft contact lens formulations are described in U.S. Pat. No. 5,710,302, WO 9421698, EP 406161, JP 2000016905, U.S. Pat. No. 5,998,498, U.S. Ser. No. 09/532,943 and U.S. Pat. No. 6,087,415. In addition, ligand monomers may be added to the formulations of commercial soft contact lenses. Examples of commercially available soft contact lenses formulations include but are not limited to, the formulations of etafilcon A, genfilcon A, lenefilcon A, polymacon, acquafilcon A, balafilcon A, and lotrafilcon A. The preferable contact lens formulations are etafilcon A, balafilcon A, and silicone hydrogels, as prepared in U.S. Pat. Nos. 5,760,100; 5,776,999; 5,849,811; 5,789,461; 5,998,498, U.S. pat. app. Ser. No. 09/532,943, a continuation-in-part of U.S. pat. app. Ser. No. 09/532,943, filed on Aug. 30, 2000, and U.S. Pat. No. 6,087,415.

Other lens forming components such as crosslinkers, UV absorbing agents, tinting agents are known in the art and need not be described here.

The type of initiator used in the present invention is not critical. Suitable intitiators include thermal initators such as lauryl peroxide, benzoyl peroxide, isopropyl percarbonate, azobisisobutyronitrile, and the like, that generate free radicals at moderately elevated temperatures, and photoinitiator systems such as aromatic alpha-hydroxy ketones, alkoxyoxybenzoins, acetophenones, acylphosphine oxides, bisacylphosphine oxides, and a tertiary amine plus a diketone, mixtures thereof and the like. Illustrative examples of photoinitiators are 1-hydroxycyclohexyl phenyl ketone, 2-hydroxy-2-methyl-1-phenyl-propan-1-one, bis(2,6-dimethoxybenzoyl)-2,4-4-trimethylpentyl phosphine oxide (DMBAPO), bis(2,4,6-trimethylbenzoyl)-phenyl phosphineoxide (Irgacure 819), 2,4,6-trimethylbenzyldiphenyl phosphine oxide and 2,4,6-trimethylbenzoyl diphenylphosphine oxide, benzoin methyl ester and a combination of camphorquinone and ethyl 4-(N,N-dimethylamino)benzoate. Commercially available visible light initiator systems include Irgacure 819, Irgacure 1700, Irgacure 1800, Irgacure 819, Irgacure 1850 (all from Ciba Specialty Chemicals) and Lucirin TPO initiator (available from BASF). Commercially available UV photoinitiators include Darocur 1173 and Darocur 2959 (Ciba Specialty Chemicals). These and other photoinitiators which may be used are disclosed in Volume III, Photoinitiators for Free Radical Cationic & Anionic Photopolymerization, 2^{nd} Edition by J. V. Crivello & K. Dietliker; edited by G. Bradley; John Wiley and Sons; New York; 1998, which is incorporated herein by reference.

The ligand monomers or their homopolymers, are mixed with the lens forming components in a diluent, prior to polymerization in an amount based on the weight percent of the initial monomer mix, including a suitable diluent if said diluent is used in the preparation of the polymer. The weight percentage of the ligand monomers can vary with the lens formulation. The maximum percentage of ligand monomers is the percentage that does not compromise the physical properties of the resulting contact lens, such as, but not limited to, modulus of the resulting lens. The minimum percentage of ligand monomers is an amount that allows the incorporation of a sufficient amount of silver into a lens to provide the desired antimicrobial effect. Preferably, about 0.01 to about 20.0 weight percent (based upon the total weight of lens forming components and ligand monomer) of ligand monomers is added, to a contact lens formulation, more preferably, about 0.01 to about 3 weight percent, and in some embodiments as little as 100 ppm to about 2000 ppm may be added.

It has been found that by controlling the polymerization or cure conditions uptake of silver may be greatly improved. Polymerization conditions sufficient to provide a ligand monomer to lens forming component reactivity ratio of greater than about 0.45 and preferably greater than about 0.5 form a lens which is capable of taking up at least 80% of a target silver concentration and preferably greater than about 85% of the target silver concentration, and in some embodiments more preferably greater than about 90% of the target silver concentration. As used herein, the term “target silver concentration” means the total amount of silver which would be predicted to be incorporated into a lens based upon the amount of ligand monomer which has been incorporated into the lens.

Kinetic models known to those skilled in the art may be used to describe the reaction rate for a given reaction component. Some of these models are described for example in Principles of Polymerization, Third Edition by George Odian, John Wiley & Sons, New York:1991, chapter 6. For example, the concentration of unreacted cystamine during the reaction can be expressed with the equation:

[CYST](t)=Res+Ae^{(-t/τ)}

where [CYST](t) is the normalized concentration of cystamine as a function of reaction time t and is expressed in units of concentration, Res is the normalized concentration of residual (unreacted) cystamine after the reaction is exhausted, A=(1−Res) is the normalized initial cystamine concentration, and τ is the exponential decay constant. The reactivity r_{CYST}=1/τ_{CYST}.

The reactivity constant, r, can be determined using experimental methods, such as those described in Example 11, below. Using data fitting software such as SigmaPlot 8.0, the normalized residual concentration of ligand monomer at each time interval is plotted versus the reaction time. An exponential trendline is fitted to the data. The exponential fit provides the reactivity constant r value, which has units of time ⁻¹. This process is repeated to determine the reactivity constant for at least one of the lens forming components, and preferably at least one major (in terms of concentration) lens forming component.

It has been found that when the polymerization conditions for the monomer mixture are selected such that the reactivity rate of the ligand monomer is close to the reactivity rate of at least one lens forming component the lenses formed therefrom display improved uptake-of silver ions. Preferably the at least one major lens forming component comprises at least about 30 weight percent of said reactive monomer mixture, and in some embodiments at least about 50 weight percent of said reactive monomer mixture. The at least one major lens forming component may be a single component, or may comprise two or more lens forming components. When the cure conditions are selected such that the ligand monomer has a reactivity rate that is close to more than one lens forming component, the lens forming components may have similar properties (such as solubility, reactivity rate, etc.) or may have different properties. As used herein, close means that the ratio of the reactivity rate of the ligand monomer to the at least one major lens forming component is at least about 0.45, preferably at least about 0.5. In some embodiments it may be preferable to have reactivity ratios of greater than about 0.6 and even greater than about 0.7.

One of skill in the art, with reference to the disclosure, including the examples of the present invention will be able to determine the appropriate set of cure conditions for a variety of systems.

The primary conditions to be controlled are cure intensity and initiator concentration. For visible light initiated systems relatively low cure intensities (such as about 1 mW/cm²) may be used so long as relatively high concentrations of initiator (at least about 1.3%) are used. Those of skill in the art will appreciate that a similar effect may be achieved by using lower amounts of photoinitiator (at least about 0.4%) with higher intensities (greater than about 6mW/cm²). Other factors, such as temperature, which change the rate of cure of the lens forming components may also be varied to achieve lower combinations of initiator concentration and cure intensity. The lens forming components and ligand monomer should be compatible at the selected reaction conditions.

Lenses prepared according to the present invention may be coated with a number of agents that are used to coat lenses. For example, the procedures, compositions, and methods of U.S. Pat. Nos. 3,854,982; 3,916,033; 4,920,184; and 5,002,794; 5,712,327; and 6,087,415 as well as WO 0127662, may be used. The lenses of this invention may be treated by other methods known in the art, such as those disclosed in U.S. Pat. Nos. 5,453,467; 5,422,402; WO 9300391; U.S. Pat. Nos. 4,973,493; and 5,350,800.

Hard contact lenses are made from polymers that include but are not limited to polymers of poly(methyl)methacrylate, silicon acrylates, fluoroacrylates, fluoroethers, polyacetylenes, and polyimides, where the preparation of representative examples may be found in U.S. Pat. No. 4,330,383. Intraocular lenses of the invention can be formed using known materials. For example, the lenses may be made from a rigid material including, without limitation, polymethyl methacrylate, polystyrene, polycarbonate, or the like, and combinations thereof. Additionally, flexible materials may be used including, without limitation, hydrogels, silicone materials, acrylic materials, fluorocarbon materials and the like, or combinations thereof. Typical intraocular lenses are described in WO 0026698; WO 0022460; WO 9929750; WO 9927978; WO 0022459. The ligand monomers may be added to hard contact lens formulations and intraocular lens formulations in the same manner and at the same percentage as described above for soft contact lenses. All of the references mentioned in this application are hereby incorporated by reference in their entirety.

As used herein, the term “silver” refers to silver metal that is incorporated into a lens. While not wanting to be bound as to the oxidation state of the silver (Ag⁰, Ag¹⁺, or Ag²⁺), that is incorporated into the lens, silver may be added to the lens by contacting the cured and hydrated lens with a silver solution such as silver nitrate in deionized water (“DI”). Other sources of silver include but are not limited to silver acetate, silver citrate, silver iodide, silver lactate, silver picrate, and silver sulfate. It will also be appreciated that other antimicrobial metal ions may be used, such as Al⁺³, Cr⁺², Cr⁺³, Cr⁶, Cd⁺¹, Cd⁺², Co⁺², Co⁺³, Ca⁺², Mg⁺², Ni⁺², Ti⁺², Ti⁺³, Ti⁺⁴, V⁺², V⁺³, V⁺⁵, Sr⁺², Fe⁺², Fe⁺³, Au⁺², Au⁺³, Au⁺¹, Ag⁺², Ag⁺¹, Pd⁺², Pd⁺⁴, Pt⁺², Pt⁺⁴, Cu⁺¹, Cu⁺², Mn⁺², Mn⁺³, Mn⁺⁴, Zn⁺² so long as the metal can be bound and released by the ligands in amounts sufficient to provide the desired level of antimicrobial efficacy and optical clarity and lack of color. Preferred other metals ions are Mg⁺², Zn⁺², Cu⁺¹, Cu⁺², Au⁺², Au⁺³, Au⁺¹, Pd⁺², Pd⁺⁴, Pt⁺², Pt⁺⁴. The part metal ion is Ag⁺¹. As above, the hydrated lens is contacted with a solution containing at least one metal salt, such as, but not limited to manganese sulfide, zinc oxide, zinc sulfide, copper sulfide, and copper phosphate.

The concentration of silver in these solutions can vary from the concentration required to add a known quantity of silver to a lens to a saturated silver solution. In order to calculate the concentration of the silver solution needed, the following calculation is used: the concentration of silver solution is equal to the desired amount of silver per lens, multiplied by the dry weight of the lens divided by the total volume of treating solution.

silver solution concentration (μg/mL)=[desired silver in lens (μg/g)×average dry lens weight(g)]/total volume of treating solution (mL)

For example, if one requires a lens containing 40 μg/g of silver, the dry weight of the lens is 0.02 g, and the vessel used to treat said lens has a volume of 3 mL, the required silver concentration would be 0.27 μg/mL.

Silver solutions containing anywhere from about 0.10 μg/mL to 0.3 grams/mL may be used depending upon the concentration of the ligand to prepare the lenses of the invention. Aside from deionized water, other liquid mediums can be used such as water, aqueous buffered solutions and organic solutions such as polyethers or alcohols. Typically, the lens is contacted with the silver solution for about 60 minutes, though the time may vary from about 1 minute to about 2 hours and at temperatures ranging from about 5° C. to about 130° C. After the silver treatment the lenses are washed with several portions of water to obtain a lens where silver is incorporated into the polymer. The amount of silver that is incorporated into the lenses ranges from about 0.001 weight % (10 ppm) to about 10 weight% (100,000 ppm), where any lens containing at least about 10 ppm has the desired antimicrobial properties. The preferred amount of silver that is incorporated into the lens is about 10 ppm to about 4,000 ppm, more preferably, 30 ppm to about 2,000 ppm, even more preferably about 30 ppm to about 1,000 ppm.

The term “antimicrobial” refers to a lens that exhibit one or more of the following properties—the inhibition of the adhesion of bacteria or other microbes to the lenses, the inhibition of the growth of bacteria or other microbes on the lenses, and the killing of bacteria or other microbes on the surface of the lenses or in a radius extending from the lenses (hereinafter adhesion of bacteria or other microbes to the lenses, the growth of bacteria or other microbes to the lenses and the presence of bacteria or other microbes on the surface of lenses is collectively referred to as “microbial production” ). The lenses of the invention inhibit the microbial production by at least 25%. Preferably, the lenses of the invention exhibit at least a 1-log reduction (≧90% inhibition) of viable bacteria or other microbes, bacteria or other microbes. Such bacteria or other microbes include but are not limited to those organisms found in the eye, particularly Pseudomonas aeruginosa, Acanthamoeba species, Staphyloccus. aureus, E. coli, Staphyloccus epidermidis, and Serratia marcesens. Preferably, said antimicrobial lens is a clear lens, that has color and clarity comparable to currently available commercial lenses such as but not limited to, etafilcon A, genfilcon A, lenefilcon A, polymacon, acquafilcon A, balafilcon A, and lotrafilcon A.

The term, “silver solution” refers to any liquid medium containing silver. The liquid medium includes but is not limited to water, deionized water, aqueous buffered solutions, alcohols, polyols, and glycols, where the preferred medium is deionized water. The silver of the solution is typically a silver salt such as silver nitrate, silver acetate, silver citrate, silver iodide, silver lactate, silver picrate, and silver sulfate. The concentration of silver in these solutions can vary from the concentration required to add a known quantity of silver to a lens to a saturated silver solution. The concentration of the silver solution needed may be calculated as described above.

Silver solutions containing anywhere from about 0.10 μg/mL to 0.3 grams/mL have been used to prepare the lenses of the invention. Aside from deionized water, other liquid mediums can be used such as water, aqueous buffered solutions and organic solutions such as polyethers, or alcohols. Typically, the lens is contacted with the silver solution for about 60 minutes, though the time may vary from about 1 minute to about 2 hours and at temperatures ranging from about 5° C. to about 130° C. In a preferred embodiment the lens is placed in the silver solution for at least about 15 minutes at a temperature between about 100 and about 150° C. In another embodiment, the silver solution is a packaging solution. The lens is placed in a package, with the packaging silver solution, the package is sealed and autoclaved. The lens may be exposed to multiple autoclaving cycles, however it has been found that excessive autoclaving may undesirably retard the release of silver from the lens. Accordingly, in some embodiments it is preferable that lenses packaged in a silver containing packaging solution be autoclaved for no more than four cycles and preferably no more than three cycles.

After the silver treatment the lenses may be washed with several portions of water to obtain a lens where silver is incorporated into the polymer.

In order to illustrate the invention the following examples are included. These examples do not limit the invention. They are meant only to suggest a method of practicing the invention. Those knowledgeable in contact lenses as well as other specialties may find other methods of practicing the invention. However, those methods are deemed to be within the scope of this invention.

Silver content of the solution after lens autoclaving was determined by Instrumental Neutron Activation Analysis “INM”. INAA is a qualitative and quantitative elemental analysis method based on the artificial induction of specific radionuclides by irradiation with neutrons in a nuclear reactor. Irradiation of the sample is followed by the quantitative measurement of the characteristic gamma rays emitted by the decaying radionuclides. The gamma rays detected at a particular energy are indicative of a particular radionuclide's presence, allowing for a high degree of specificity. Becker, D. A.; Greenberg, R. R.; Stone, S. F. J. Radioanal. Nucl. Chem. 1992,160(1), 41-53; Becker, D. A.; Anderson, D. L.; Lindstrom, R. M.; Greenberg, R. R.; Garrity, K. M.; Mackey, E. A. J. Radioanal. Nucl. Chem. 1994,179(1),149-54. The INAA procedure used to quantify silver content in contact lens material uses the following two nuclear reactions:

1. In the activation reaction, ¹¹⁰Ag is produced from stable ¹⁰⁹Ag (isotopic abundance=48.16%) after capture of a radioactive neutron produced in a nuclear reactor.

2. In the decay reaction, ¹¹⁰Ag (τ^{1/2}=24.6 seconds) decays primarily by negatron emission proportional to initial concentration with an energy characteristic to this radio-nuclide (657.8 keV).

The gamma-ray emission specific to the decay of ¹¹⁰Ag from irradiated standards and samples are measured by gamma-ray spectroscopy, a well-established pulse-height analysis-technique, yielding a measure of the concentration of the analyte.

### EXAMPLES

The following abbreviations were used in the examples

CYST =N,N′-bis (acryloyl)cystamine (CYST) commercially supplied from Fluka

MAA=methacrylic acid;

HEMA=hydroxyethyl methacrylate

Blue HEMA=the reaction product of reactive blue number 4 and HEMA as described in Example 4 of U.S. Pat. No. 5,944,853

EGDMA=ethyleneglycol dimethacrylate

TMPTMA=trimethyloyl propane trimethacrylate

Norbloc 7966=a UV blocking component consisting of 2-(2′-hydroxy-5′-methacrylyloxyethylphenyl)-2H-benzotriazole

Irgacure 1850=1:1 (w/w) blend of 1-hydroxycyclohexyl phenyl ketone and bis (2,6-dimethyoxybenzoyl)-2,44-trimethylpentyl phosphine oxide, commercially available from Ciba Specialty Chemicals Inc

### Examples 1-3

Monomer mixes were formed from the components listed in Table 1 below, by blending the 50 wt % of the listed formulations with 50 wt % glycerin boric acid ester. All amounts are in weight %.

TABLE I

| Component | Ex. 1 | Ex. 2 | Ex. 3 |
|---|---|---|---|
| MAA | 1.95 | 1.94 | 1.94 |
| EGDMA | 0.78 | 0.77 | 0.77 |
| HEMA | 95.76 | 95.34 | 94.92 |
| TMPTMA | 0.10 | 0.10 | 0.09 |
| Irgacure 1850 | 0.45 | 0.90 | 1.33 |
| Norblock 7966 | 0.96 | 0.95 | 0.95 |
| Blue HEMA | 0.02 | 0.02 | 0.0 |

To each of the monomer mixes was added 12,000 ppm N,N′-bis (acryloyl)cystamine (CYST). The resultant mix was stirred for 40-75 minutes at about 350 rpm at approximately 25+/−5° C. to ensure a homogeneous mixture. The monomer mix was degassed at 40+/−3 mm Hg for 30-35 minutes. In Examples 2 and 3, the additional photoinitiator was added prior to the CYST.

### Examples 4-10

Contact lenses were formed by adding about 0.10 g of the monomer mix to the cavity of an eight cavity lens mold of the type described in U.S. Pat. No. 4,640,489 and curing for 1200 sec. Polymerization occurred under a nitrogen purge and was photoinitiated with visible light generated with a Philips TL 20W/03T fluorescent bulb at two different light intensities, 1 mW/cm², and 6 mW/cm². After curing, the molds were opened, and the lenses were released in distilled, deinoized water containing 800 ppm Tween 80 and 170 ppm ethylenediaminetertracarboxylic acid (EDTA), then leached in distilled, deionized water to remove any residual monomers and diluent. Finally the lenses were equilibrated in physiological, borate-buffered, silver nitrate containing, saline packing solution which contained approximately 0.7 microgram/ml. of silver while in a polypropylene blister package. The lens in silver containing packing solution were autoclaved for 30 minutes at about 122.5° C. following an approximately 5 minute ramp up from room temperature at approximately 20° per minute. Subsequently, the lenses were ramped down to 40° C. 10° C. per minute.

After autoclaving, the lenses were analyzed for silver using INAA. At least four lenses were sampled for each analysis and the results are reported as an average in Table 2, below.

TABLE 2

| | Residual | Initiator | Intensity | Belt spd | Ag target | % |
|---|---|---|---|---|---|---|
| Ex # | CYST (ppm) | (ppm) | (mW) | (fpm) | (ppm) | target |
| 4 | 265 | 0.45 | 1 | 4 | 70 | 62 |
| 5 | 340 | 0.45 | 1 | 4 | 75 | 73 |
| 6 | 52 | 0.9 | 1 | 2.5 | 100 | 71 |
| 7 | 139 | 0.9 | 1 | 2.5 | 75 | 70 |
| 8 | 52 | 1.35 | 1 | 2 | 75 | 100 |
| 9 | 217 | 0.45 | 6 | 3.2 | 75 | 81 |
| 10 | 124 | 0.9 | 6 | 3.2 | 75 | 100 |

It can be seen from the data in Table 2, that at low intensity (1 mW/cm²), both low and intermediate concentrations of initiator fail to provide complete incorporation of silver, with only 61.9 to 72.9% silver incorporation. However, at initiator concentrations of 1.35 (Example 8, 3× the lowest value) complete incorporation of silver is achieved. High intensity cure (6 mW/cm²) provides improved percent incorporation, but both elevated intensity and initiator concentration are required to insure complete (100%) incorporation (Example 10). Table 2 also shows that the amount of residual CYST not incorporated into the polymer has no effect on the efficiency of silver uptake by the lens (compare Example 4 to Example 5 and Example 6 to Example 7). Similarly, the exposure time (belt speed) also has no effect on the efficiency of incorporation of silver into the lens.

### Example 11

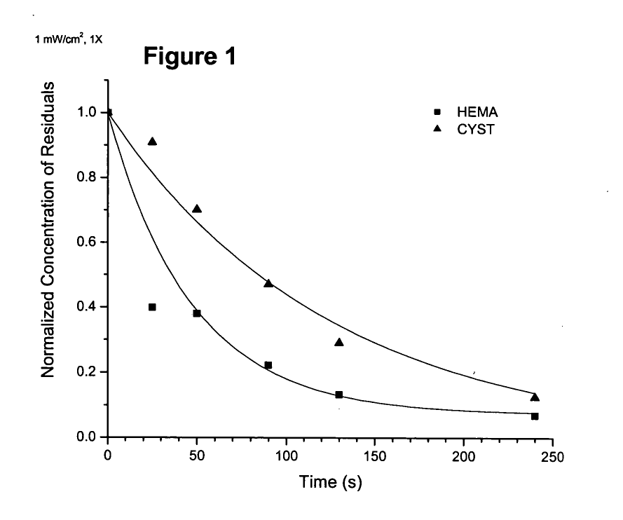

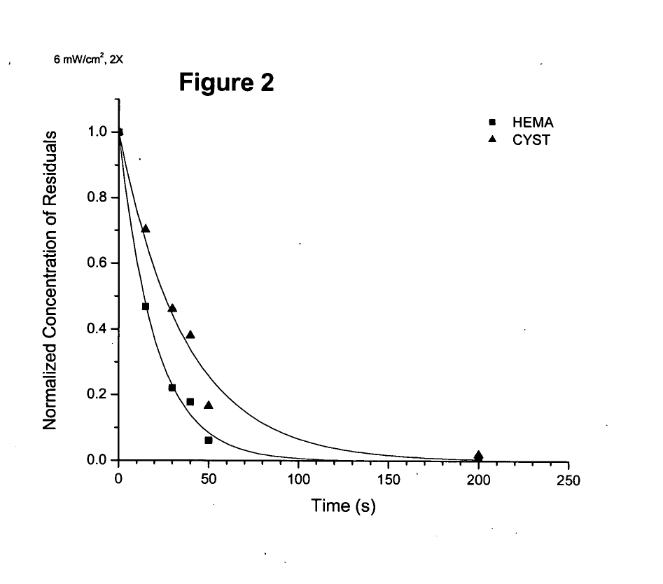

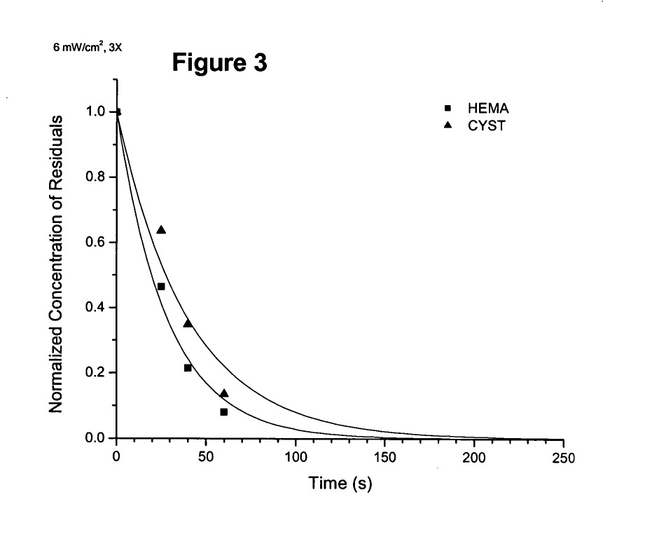

The concentrations of unreacted HEMA and CYST remaining in 500 μm thick films of the various formulations were measured by liquid chromatography after exposing them to radiation at 420 nm (20 nm FWHM) as a function of light intensity, photoinitiator concentration and exposure time. The normalized residual concentrations of HEMA and CYST at various reaction times are plotted in FIGS. 1-3.

The residual concentrations were normalized and fit to a first order exponential decay equation,

[component](t)=Res+A exp(−t/τ)

where [component](t) is the concentration of the component as a function of exposure time t, Res is the concentration of residual (unreacted) component after the reaction is exhausted, A(=1−Res) is the normalized initial concentration, and τ is the exponential decay constant. The reactivity r_{component}=1/τ_{component}, at each initiator concentration/cure intensity condition was calculated. The results are listed in Table 3, below.

TABLE 3

| | [Initiator] | τ_{component} at | | |
|---|---|---|---|---|
| Monomer | (wt %) | 1 mW/cm² | 6 mW/cm² | 18.5 mW/cm² |
| HEMA | 0.45 | 45.1 | 31.5 | |
| HEMA | 0.90 | 33.4 | 20.3 | |
| HEMA | 1.35 | 62.4 | 28.8 | 18.3 |
| CYST | 0.45 | 121.9 | 68.5 | |
| CYST | 0.90 | 79.4 | 36.9 | |
| CYST | 1.35 | 85.5 | 39.7 | 29.6 |

The reactivity ratio, RR=r_{CYST}/r_{HEMA} and was calculated at each initiator/intensity point listed in Table 3. The ratios are shown in Table 4, below.

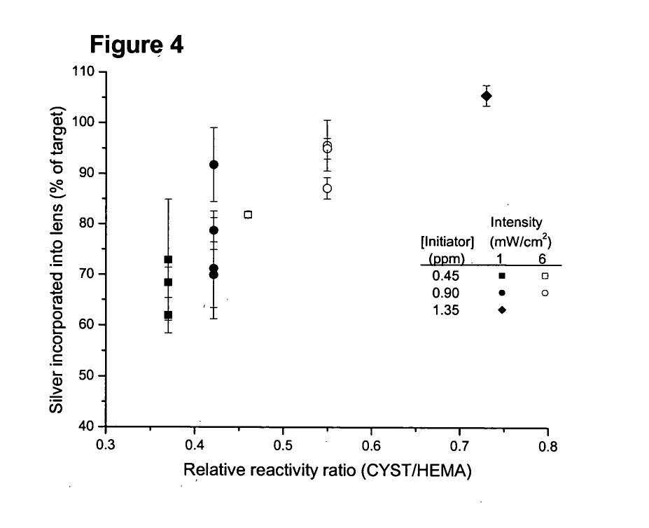

Lenses made from the same formulations and under a similar set of conditions (intensity, temperature) were treated with a silver nitrate-containing saline solution. The amounts of silver incorporated into the lens were measured by INAA. FIG. 4 shows the efficiency of silver incorporation as a function of the relative reactivity ratio.

TABLE 4

| | RR at | | |
|---|---|---|---|
| [Initiator] (%) | 1 mW/cm² | 6 mW/cm² | 18.5 mW/cm² |
| 0.45 | 0.37 | 0.46 | NM |
| 0.90 | 0.42 | 0.55 | NM |
| 1.35 | 0.73 | 0.73 | 0.62 |

NM = not measured

The reactivity ratios from Table 4, above were plotted against the amount of silver incorporated into the lens, expressed as a percentage of the target silver concentration. FIG. 4 clearly shows that when cure conditions which provide reactivity ratios of greater than about 0.45 are used, lenses displaying at least at 80% silver incorporation are formed. When cure conditions which provide reactivity ratios of greater than about 0.5 are used, lenses displaying at least about 85% silver incorporation are formed.

What is claimed is:

1. A method comprising the steps of
(a) curing a reactive monomer mix comprising at least one lens forming component and at least one ligand monomer under conditions sufficient to provide a reactivity ratio of the ligand monomer to at least one major lens forming component of at least about 0.45 lens; and
(b) treating said lens with a silver solution to form an antimicrobial lens comprising silver in an amount which is at least about 80% of target silver concentration, where the ligand monomer is of Formulae 1, 11, III or IV, 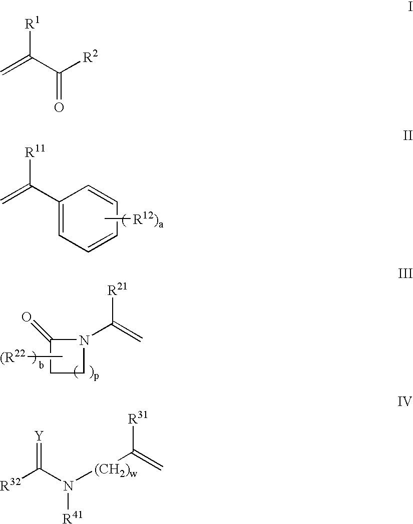
wherein
R¹ is hydrogen or C₁₋₆alkyl;
R is —OR³, —NH—R³, —S—(CH₂)_{d}—R³,or —(CH₂)_{d}—R³, wherein
d is 0-8;
R³ is substituted C₁₋₆alkyl
where the alkyl substituents are selected from one or more members of the group consisting of carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR⁴R⁵)_{q}—(CHR⁶)ₘ—SO₃H
wherein R⁴, R⁵, and R⁶ are independently selected from the group consisting of hydrogen, halogen, hydroxyl, and C₁₋₆alkyl,
q is 1-6, and
m is 0-6;
—(CH₂)ₙ—S—S—(CH₂)ₓNH—C(O)CR⁷CH₂,
wherein R⁷ is hydrogen or C₁₋₆alkyl,
n is 1-6, and
x is 1-6;
—(CR⁸R⁹)ₜ—(CHR¹⁰)ᵤ—P(O)(OH)₂
wherein R⁸, R⁹, and R¹⁰ are independently selected from the group consisting of hydrogen, halogen, hydroxyl, and C₁₋₆alkyl,
t is 1-6, and
u is 0-6;
phenyl, benzyl, pyridinyl, pyrimidinyl, pyrazinyl, benzimidazolyl, benzothiazolyl, benzotriazolyl, naphthaloyl, quinolinyl, indolyl, thiadiazolyl, triazolyl, 4-methylpiperidin-1-yl, 4-methylpiperazin-1-yl, substituted phenyl, substituted benzyl, substituted pyridinyl, substituted pyrimidinyl, substituted pyrazinyl, substituted benzimidazolyl, substituted benzothiazolyl, substituted benzotriazolyl, substituted naphthaloyl, substituted quinolinyl, substituted indolyl, substituted thiadiazolyl, substituted triazolyl, substituted 4-methylpiperidin-1-yl; or
substituted 4-methylpiperazin-1-yl,
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine,
N-(2-aminopyrimidine)sulfonyl,
N-(aminopyridine)sulfonyl, N-(aminopyrazine)sulfonyl,
N-(2-aminopyrimidine)carbonyl,
N-(aminopyridine)carbonyl, N-(aminopyrazine)carbonyl,
N-(2-aminopyrimidine)phosphonyl,
N-(2-aminopyridine)phosphonyl,
N-(aminopyrazine)phosphonyl,
N-(aminobenzimidazolyl)sulfonyl,
N-(aminobenzothiazolyl)sulfonyl,
N-(aminobenzotriazolyl)sulfonyl,
N-(aminoindolyl)sulfonyl, N-(aminothiazolyl)sulfonyl,
N-(aminotriazolyl)sulfonyl,
N-(amino-4-methylpiperidinyl)sulfonyl,
N-(amino-4-methylpiperazinyl)sulfonyl,
N-(aminobenzimidazolyl)carbonyl,
N-(aminobenzothiazolyl)carbonyl,
N-(aminobenzotriazolyl)carbonyl,
N-(aminoindolyl)carbonyl, N-(aminothiazolyl)carbonyl,
N-(aminotriazolyl)carbonyl,
N-(amino-4-methylpiperidinyl)carbonyl,
N-(amino-4-methylpiperazinyl)carbonyl,
N-(2-aminobenzimidazolyl)phosphonyl,
N-(2-aminobenzothiazolyl)phosphonyl,
N-(2-aminobenzotriazolyl)phosphonyl,
N-(2-aminoindolyl)phosphonyl,
N-(2-aminothiazolyl)phosphonyl,
N-(2-aminotriazolyl)phosphonyl,
N-(amino-4-methylpiperidinyl) phosphonyl,
N-(amino-4-methylpiperazinyl) phosphonyl, acetamide,
nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrite;
a is 1-5;
R¹¹ is hydrogen or C₁₋₆alkyl;
R¹² is hydroxyl, sulfonic acid, phosphonic acid, carboxylic acid, acetamide, thioC₁₋₆alkylcarbonyl, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, —OR¹³, —NH—R¹³, —S—(CH₂)_{d}—R¹³, —(CH₂)_{d}—R¹³, —C(O)NH—(CH₂)_{d}—R¹³, —C(O)—(CH₂)_{d}—R¹³, substituted C₁₋₆alkyldisulfide, substituted phenyldisulfide, substituted C₁₋₆alkylurea, substituted phenylurea, substituted phenylthiourea or substituted C₁₋₆alkylthiourea wherein the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile; where
d is 0-8;
R¹³ is thioC₁₋₆alkylcarbonyl;
substituted C₁₋₆alkyl
where the alkyl substituents are selected from one or more members of the group consisting of hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR¹⁴R¹⁵)_{q}—(CHR¹⁶)ₘ—SO₃H
where R¹⁴, R¹⁵, and R¹⁶ are independently selected from the group consisting of hydrogen, halogen, hydroxyl, and C₁₋₆alkyl,
q is 1-6, and
m is 0-6;
—(CH₂)ₙ—S—S—(CH₂)ₓNH—C(O)CR¹⁷CH₂,
where R¹⁷ is hydrogen or C₁₋₆alkyl,
n is 1-6, and
x is 1-6;
—(CR¹⁸ R¹⁹)ₜ—(CHR²⁰)ᵤ—P(O)(OH)₂
where R¹⁸, R¹⁹, and R²⁰ are independently selected from the group consisting of hydrogen, halogen, hydroxyl, and C₁₋₆alkyl,
t is 1-6, and
u is 0-6;
phenyl; benzyl; pyridinyl; pyrimidinyl; pyrazinyl;
benzimidazolyl; benzothiazolyl; benzotriazolyl;
naphthaloyl; quinolinyl; indolyl; thiadiazolyl; triazolyl;
4-methylpiperidin-1-yl; 4-methylpiperazin-1-yl;
substituted phenyl; substituted benzyl; substituted pyridinyl; substituted pyrimidinyl; substituted pyrazinyl;
substituted benzimidazolyl; substituted benzothiazolyl;
substituted benzotriazolyl; substituted naphthaloyl;
substituted quinolinyl; substituted indolyl; substituted thiadiazolyl; substituted triazolyl; substituted 4-methylpiperidin-1-yl;
or substituted 4-methylpiperazin-1-yl
wherein the substituents are selected from one or more members of the group consisting of C₁₋₆alkyl, haloC₁-alkyl, halogen, sulfonic acid, phosphonic acid, hydroxyl, carboxylic acid, amine, amidine,
N-(2-aminopyrimidine)sulfonyl,
N-(aminopyridine)sulfonyl, N-(aminopyrazine)sulfonyl,
N-(2-aminopyrimidine)carbonyl,
N-(aminopyridine)carbonyl, N-(aminopyrazine)carbonyl,
N-(2-aminopyrimidine)phosphonyl,
N-(2-aminopyridine)phosphonyl,
N-(aminopyrazine)phosphonyl,
N-(aminobenzimidazolyl)sulfonyl,
N-(aminobenzothiazolyl)sulfonyl,
N-(aminobenzotriazolyl)sulfonyl,
N-(aminoindolyl)sulfonyl, N-(aminothiazolyl)sulfonyl,
N-(aminotriazolyl)sulfonyl,
N-(amino-4-methylpiperidinyl)sulfonyl,
N-(amino-4-methylpiperazinyl)sulfonyl,
N-(aminobenzimidazolyl)carbonyl,
N-(aminobenzothiazolyl)carbonyl,
N-(aminobenzotriazolyl)carbonyl,
N-(aminoindolyl)carbonyl, N-(aminothiazolyl)carbonyl,
N-(aminotriazolyl)carbonyl,
N-(amino-4-methylpiperidinyl)carbonyl,
N-(amino-4-methylpiperazinyl)carbonyl,
N-(2-aminobenzimidazolyl)phosphonyl,
N-(2-aminobenzothiazolyl)phosphonyl,
N-(2-aminobenzotriazolyl)phosphonyl,
N-(2-aminoindolyl)phosphonyl,
N-(2-aminothiazolyl)phosphonyl,
N-(2-aminotriazolyl)phosphonyl,
N-(amino-4-methylpiperidinyl) phosphonyl,
N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
b is 1-5; p is 1-5; R²¹ is hydrogen;
R²² is hydroxyl, sulfonic acid, phosphonic acid, carboxylic acid, thioC₁₋₆alkylcarbonyl, thioC₁₋₆alkylaminocarbonyl, C₁₋₆alkyldisulfide, phenyldisulfide, —C(O)N H(CH₂)₁₋₆—SO₃H, —C(O)NH(CH₂)₁₋₆—P(O)(OH)₂, —OR²³, −NH—R²³, —C(O)NH—(CH₂)_{d}—R²³—S—(CH₂)_{d}—R²³, —(CH₂)_{d}—R²³, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted, C₁₋₆alkylthiourea substituted phenylurea or substituted phenylthiourea wherein the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile,
where
d is 0-8;
R²³ is thioC₁₋₆alkylcarbonyl,
C₁₋₆alkyl,
substituted C₁₋₆alkyl
where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR²⁴R²⁵)_{q}—(CHR²⁶)ₘ—SO₃H
where R²⁴, R²⁵, and R²⁶ are independently selected from the group consisting of hydrogen, halogen, hydroxyl, and C₁₋₆alkyl,
q is 1-6, and m is 0-6
—(CH₂)ₙ—S—S—(CH₂)ₓNH—C(O)CR²⁷CH₂,
where R is hydrogen or C₁₋₆alkyl,
n is 1-6, and x is 1-6;
—(CR²⁸R²⁹)ₜ(CHR³⁰)ᵤ—P(O)(OH)₂
where R²⁸, R²⁹, and R³⁰ are independently selected from the group consisting of hydrogen, halogen, hydroxyl, and C₁₋₆alkyl,
t is 1-6, and u is 0-6;
phenyl; benzyl; pyridinyl; pyrimidinyl; pyrazinyl;
benzimidazolyl; benzothiazolyl; benzotriazolyl; naphthaloyl;
quinolinyl; indolyl; thiadiazolyl; triazolyl;
4-methylpiperidin-1-yl; 4-methylpiperazin-1-yl;
substituted phenyl; substituted benzyl; substituted pyridinyl; substituted pyrimidinyl; substituted pyrazinyl;
substituted benzimidazolyl; substituted benzothiazolyl;
substituted benzotriazolyl; substituted naphthaloyl;
substituted quinolinyl; substituted indolyl; substituted thiadiazolyl; substituted triazolyl; substituted 4-methylpiperidin-1-yl;
or substituted 4-methylpiperazin-1-yl,
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine,
N-(2-aminopyrimidine)sulfonyl,
N-(aminopyridine)sulfonyl, N-(aminopyrazine)sulfonyl,
N-(2-aminopyrimidine)carbonyl,
N-(aminopyridine)carbonyl, N-(aminopyrazine)carbonyl,
N-(2-aminopyrimidine)phosphonyl,
N-(2-aminopyridine)phosphonyl,
N-(aminopyrazine)phosphonyl,
N-(aminobenzimidazolyl)sulfonyl,
N-(aminobenzothiazolyl)sulfonyl,
N-(aminobenzotriazolyl)sulfonyl,
N-(aminoindolyl)sulfonyl, N-(aminothiazolyl)sulfonyl,
N-(aminotriazolyl)sulfonyl,
N-(amino-4-methylpiperidinyl)sulfonyl,
N-(amino-4-methylpiperazinyl)sulfonyl,
N-(aminobenzimidazolyl)carbonyl,
N-(aminobenzothiazolyl)carbonyl,
N-(aminobenzotriazolyl)carbonyl,
N-(aminoindolyl)carbonyl, N-(aminothiazolyl)carbonyl,
N-(aminotriazolyl)carbonyl,
N-(amino-4-methylpiperidinyl)carbonyl,
N-(amino-4-methylpiperazinyl)carbonyl,
N-(2-aminobenzimidazolyl)phosphonyl,
N-(2-aminobenzothiazolyl)phosphonyl,
N-(2-aminobenzotriazolyl)phosphonyl,
N-(2-aminoindolyl)phosphonyl,
N-(2-aminothiazolyl)phosphonyl,
N-(2-aminotriazolyl)phosphonyl,
N-(amino-4-methylpiperidinyl) phosphonyl,
N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
w is 0-1;
Y is oxygen or sulfur; R³1 is hydrogen or C₁₋₆alkyl;
R³² is hydroxyl, sulfonic acid, phosphonic acid, carboxylic acid, thioC₁₋₆alkylcarbonyl, thioC₁₋₆alkylaminocarbonyl, —C(O)NH—(CH₂)_{d}—R³³, —O—R³³, −NH—R³³, —S—(CH₂)_{d}—R³³, —(CH2)_{d}—R³³, C₁₋₆alkyldisulfide, phenyldisulfide, urea, C₁₋₆alkylurea, phenylurea, thiourea, C₁₋₆alkylthiourea, phenylthiourea, C₁₋₆alkylamine, phenylamine, substituted C₁₋₆alkyldisulfide, substituted phenyldisulfide, substituted phenylurea, substituted C₁₋₆alkylamine, substituted phenylamine, substituted phenylthiourea, substituted C₁₋₆alkylurea or substituted C₁₋₆alkylthiourea wherein the substitutents are selected from the group consisting of C₁₋₆alkyl, haloC₁₋₆alkyl, halogen, hydroxyl, carboxylic acid, sulfonic acid, phosphonic acid, amine, amidine, acetamide, and nitrile where
d is 0-8;
R³³ is thioC₁₋₆alkylcarbonyl, C₁₋₆alkyl, substituted C₁₋₆alkyl where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea or substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR³⁴R³⁵)_{q}—(CHR³⁶)ₘ—SO₃H
where R³⁴, R³⁵, and R³⁶ are independently selected from the group consisting of hydrogen, halogen, hydroxyl, and C₁₋₆alkyl,
q is 1-6, and m is 0-6;
—(CH₂)ₙ—S—S—(CH₂)ₓNH—C(O)CR³⁷CH₂,
where R³⁷ is hydrogen or C₁₋₆alkyl,
n is 1-6, and x is 1-6;
—(CR³⁸R³⁹)ₜ—(CHR⁴⁰)ᵤ—P(O)(OH)₂
where R³⁸, R³⁹, and R⁴⁰ are independently selected from the group consisting of hydrogen, halogen, hydroxyl, and C₁₋₆alkyl,
t is 1-6, and u is 0-6;
phenyl; benzyl; pyridinyl; pyrimidinyl; pyrazinyl;
benzimidazolyl; benzothiazolyl; benzotriazolyl;
naphthaloyl; quinolinyl; indolyl; thiadiazolyl;
triazolyl; 4-methylpiperidin-1-yl; 4-methylpiperazin-1-yl;
substituted phenyl; substituted benzyl; substituted pyridinyl; substituted pyrimidinyl; substituted pyrazinyl;
substituted benzimidazolyl; substituted benzothiazolyl;
substituted benzotriazolyl; substituted naphthaloyl;
substituted quinolinyl; substituted indolyl;
substituted thiadiazolyl; substituted triazolyl;
substituted 4-methylpiperidin-1-yl; or
substituted 4-methylpiperazin-1-yl,
wherein the substituents are selected from one or more members of the group consisting of C₁₋₆alkyl, haloC₁-alkyl, halogen, sulfonic acid, phosphonic acid, hydroxyl, carboxylic acid, amine, amidine,
N-(2-aminopyrimidine)sulfonyl,
N-(aminopyridine)sulfonyl, N-(aminopyrazine)sulfonyl,
N-(2-aminopyrimidine)carbonyl,
N-(aminopyridine)carbonyl, N-(aminopyrazine)carbonyl,
N-(2-aminopyrimidine)phosphonyl,
N-(2-aminopyridine)phosphonyl,
N-(aminopyrazine)phosphonyl,
N-(aminobenzimidazolyl)sulfonyl,
N-(aminobenzothiazolyl)sulfonyl,
N-(aminobenzotriazolyl)sulfonyl,
N-(aminoindolyl)sulfonyl, N-(aminothiazolyl)sulfonyl,
N-(aminotriazolyl)sulfonyl,
N-(amino-4-methylpiperidinyl)sulfonyl,
N-(amino-4-methylpiperazinyl)sulfonyl,
N-(aminobenzimidazolyl)carbonyl,
N-(aminobenzothiazolyl)carbonyl,
N-(aminobenzotriazolyl)carbonyl,
N-(aminoindolyl)carbonyl, N-(aminothiazolyl)carbonyl,
N-(aminotriazolyl)carbonyl,
N-(amino-4-methylpiperidinyl)carbonyl,
N-(amino-4-methylpiperazinyl)carbonyl,
N-(2-aminobenzimidazolyl)phosphonyl,
N-(2-aminobenzothiazolyl)phosphonyl,
N-(2-aminobenzotriazolyl)phosphonyl,
N-(2-aminoindolyl)phosphonyl,
N-(2-aminothiazolyl)phosphonyl,
N-(2-aminotriazolyl)phosphonyl,
N-(amino-4-methylpiperidinyl) phosphonyl,
N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
R⁴¹ is hydrogen, C₁₋₆alkyl, phenyl, C₁₋₆alkylcarbonyl, phenylcarbonyl, substituted C₁₋₆alkyl, substituted phenyl, substituted C₁₋₆alkylcarbonyl or substituted phenylcarbonyl,
wherein
the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile.

2. The method of claim 1 wherein said ratio is at least about 0.5.

3. The method of claim 1 wherein the lens comprises silver in an amount which is at least about 90% of the target silver concentration.

4. The method of claim 1 wherein said at least one lens forming component comprises at least about 30 weight percent of said reactive monomer mixture.

5. The method of claim 1 wherein said at least one lens forming component comprises at least about 50 weight percent of said reactive monomer mixture.

6. The method of claim 4 wherein said at least one lens forming component comprises at least two lens forming components having similar solubilities.

7. The method of claim 1 wherein the ligand monomer is a monomer of Formula I and,
R¹ is hydrogen or C₁₋₃alkyl;
R² is NH—R³;
d is 0;
R³ is substituted phenyl, —(CR⁴R⁵)_{q}—(CHR⁶)ₘ—SO₃H,
—(CR⁸R⁹)ₜ—(CHR¹⁰)ᵤ—P(O)(OH)₂ or
—(CH₂)ₙ—S—S—(CH₂)ₓNH—C(O)CR⁷CH₂;
R⁴⁻⁶ are independently selected from the group consisting of hydrogen or C, ₃alkyl;
q is 1-3; m is 1-3;
R⁷⁻¹⁰ are independently selected from the group consisting of hydrogen or C₁₋₃alkyl;
t is 1-3; u is 1-3; n is 2-4; and x is 2-4.

8. The method of claim 1 wherein the lens is a soft contact lens.

9. The method of claim 1 wherein the lens comprises about 0.01 to about 20 weight percent ligand monomer.

10. The method of claim 1 wherein the lens comprises about 0.01 to about 3 weight percent ligand monomer.

11. The method of claim 1 wherein the lens comprises about 100 to about 2000 ppm ligand monomer.

12. The method of claim 1 wherein the lens is a silicone hydrogel.

13. The method of claim 1 wherein, the lens comprises a formulation selected from the group consisting of etafilcon A, balafilcon, A, acquafilcon A, lenefilcon A, galyfilcon A, senofilcon A and lotrafilcon A.

14. The method of claim 1 wherein,
R¹ is hydrogen or methyl;
R² is NH—R³;
R³ is —(CR⁴R⁵)_{q}—(CHR⁶)ₘ—SO₃H, —(CR⁸R⁹)ₜ13 (CHR¹⁰)ᵤ—P(O)(OH)₂ or
—(CH₂)ₙ—S—S—(CH₂)ₓNH—C(O)CHR⁷CH₂;
R⁴⁻⁶ are independently hydrogen or methyl;
q is 1-2; m is 1-2;
R⁷ is hydrogen;
R⁸⁻¹⁰ are independently hydrogen or methyl;
t is 1; u is 1-2; n is 2-3; and x is 2-3.

15. The method of claim 1 wherein the ligand monomer is selected from the group consisting of

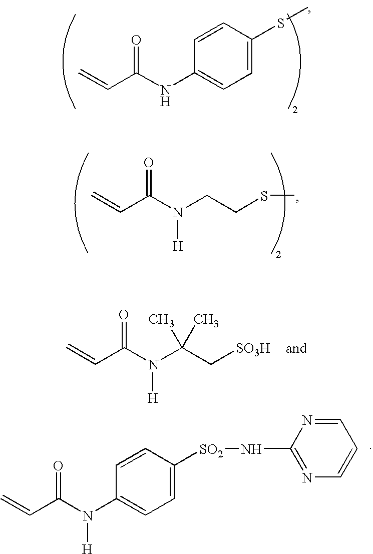

16. The method of claim 1 wherein the antimicrobial lens comprises about 10 ppm to about 4,000 ppm silver.

17. The method of claim 1 wherein the antimicrobial lens comprises about 30 ppm to about 2000 ppm silver.

18. The method of claim 1 wherein the antimicrobial lens comprises about 30 ppm to about 1000 ppm silver.

19. The method of claim 1 wherein the lens is a silicone hydrogel and the ligand monomer is

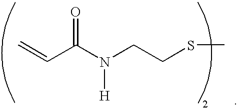

20. The method of claim 19 wherein silver is present at about 30 ppm to about 2000 ppm and the ligand monomer is present at about 0.01 to about 3 weight percent.

21. The method of claim 13 wherein the ligand monomer is

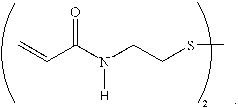

22. The method of claim 21 wherein silver is present in the antimicrobial lens at about 30 ppm to about 2000 ppm and the ligand monomer is present at about 0.01 to about 3 weight percent.

23. The method of claim 21 wherein the lens formulation is etafilcon A or acquafilcon A.

24. The method of claim 1 wherein the silver solution is aqueous silver nitrate having a concentration of about 0.1 μg/mL to about 0.3 g/mL.

25. The method of claim 1 wherein, treating comprises soaking the lens with or in a silver solution.

26. The method of claim 25 wherein, the lens is soaked in the silver solution for about 2 minutes to about 2 hours.

27. The method of claim 1 wherein, treating comprises storing the lens in the silver solution for about 20 minutes to about 5 years.

28. The method of claim 1 wherein said monomer mix further comprises at least one initiator.

29. The method of claim 28 wherein said initiator comprises at least one photoinitiator.

30. The method of claim 29 wherein the curing step comprises an initiator concentration and light intensity sufficient to provide the reactivity ratio of at least about 0.45.

31. The method of claim 30 wherein the initiator concentration is at least about 0.4 weight % and said intensity is at least about 4 mW/cm².

32. The method of claim 30 wherein the initiator concentration is at least about 0.9 weight % and said intensity is at least about 1 mW/cm².

33. The method of claim 30 wherein the initiator concentration is at least about 0.4 weight % and said intensity is at least about 6 mW/cm².

34. The method of claim 30 wherein the initiator concentration is at least about 0.9 weight % and said intensity is at least about 4 mW/cm².

35. The method of claim 30 wherein the initiator concentration about 0.4 to about 2 weight % and said intensity is at least about 4 mW/cm².

36. The method of claim 1 wherein said ligand monomer is selected from the monomers of Formula II.

37. The method of claim 36 wherein,
a is 1-2,
R¹¹ is hydrogen or C₁₋₃alkyl,
R¹² is sulfonic acid, carboxylic acid, phosphonic acid,
C₁₋₆alkyldisulfide, C₁₋₆alkylsulfide, phenyldisulfide, substiuted phenyldisulfide or NH—R¹³,
R¹³ is thioC₁₋₆alkylcarbonyl.

38. The method of claim 36 wherein the monomer of Formula II is selected from the group consisting of

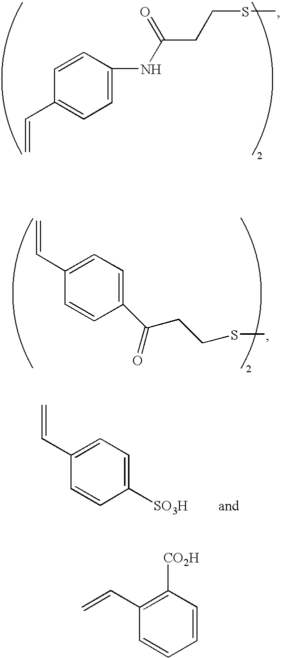

39. The method of claim 1 wherein said ligand monomer is selected from the group consisting of monomers of Formula III.

40. The method of claim 39 wherein,
p is 1-3;
b is 1-2;
R²¹ is hydrogen;
R²² is sulfonic acid, phosphonic acid, carboxylic acid,
thioC₁₋₆alkylcarbonyl, thioC₁₋₆alkylaminocarbonyl, C₁₋₆alkyldisulfide, C₁₋₆alkylsulfide, phenyldisulfide, substiuted phenyldisulfide,
H₃OS—(CH₂)₁₋₆NHC(O) or
(HO)₂(O)P—(CH₂)₁₋₆NHC(O)—.

41. The method of claim 39 wherein the monomer of Formula III is selected from the group consisting of

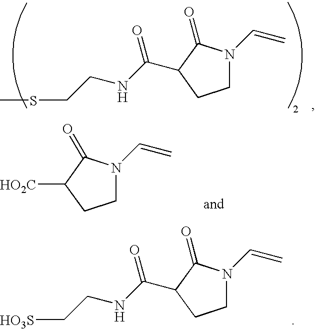

42. The method of claim 1 wherein the ligand monomer is selected from the group consisting of monomers of Formula IV.

43. The method of claim 42 wherein,
w is 0-1; R³¹ is hydrogen;
R³² is amine, C₁₋₃alkylamine, phenylamine, substituted phenylamine;
thioC₁₋₃alkylcarbonyl; and R⁴¹ is hydrogen.

44. The method of claim 42 wherein the ligand monomer is selected from the group consisting of [chemical structure]
